(19) United States
(12) Patent Application Publication
(10) Pub. No.: US 2025/0057755 A1
(43) Pub. Date: Feb. 20, 2025

(54) COMPOSITION FOR KERATIN FIBERS

(71) Applicant: L'Oreal, Paris (FR)

(72) Inventors: Takuya HARA, Kawasaki-shi (JP); Adelino NAKANO, Kawasaki-shi (JP); Akihiro KASAI, Kawasaki-shi (JP); Mathilde GHAFAR, Kawasaki-shi (JP)

(21) Appl. No.: 18/716,488

(22) PCT Filed: Nov. 25, 2022
(86) PCT No.: PCT/JP2022/044492

§ 371 Date: Jun. 4, 2024

(30) Foreign Application Priority Data
Dec. 8, 2021 (JP) 2021-199318
Jan. 13, 2022 (FR) 2200247

(51) Int. Cl. A61K 8/97; A61K 8/31; A61K 8/37; A61Q 5/08; A61Q 5/10

(52) U.S. Cl. CPC A61K 8/97; A61K 8/31; A61K 8/37; A61Q 5/08; A61Q 5/10; A61K 2800/592; A61K 2800/805

(57) ABSTRACT

The present invention relates to a composition for keratin fibers, comprising: (a) at least one biodegradable liquid fatty material; (b) at least one alkaline agent; (c) at least one thickener; and (d) water, wherein the amount of the (a) biodegradable liquid fatty material(s) is 10% by weight or more, preferably 30% by weight or more, and more preferably 50% by weight or more, relative to the total weight of the composition. The composition according to the present invention can be used for oxidative dyeing or bleaching of keratin fibers such as hair.

### TECHNICAL FIELD

The present invention relates to a composition for keratin fibers, in particular, a composition for bleaching or dyeing keratin fibers such as hair.

### BACKGROUND ART

The dyeing of keratin fibers, in particular hair, using dyeing compositions containing oxidative coloring precursors, generally called oxidative bases, such as ortho- or para-phenylenediamines, ortho- or para-aminophenols and heterocyclic compounds is well known. These oxidative bases are generally combined with couplers. These oxidative bases and couplers are colorless or weakly colored compounds. However, when combined with oxidizing agents, they can provide colored dye molecules through an oxidative condensation process.

This type of coloring by oxidation, i.e., oxidative dyeing, makes it possible to get colors with very high visibility, coverage of white hair, and a wide variety of shades. Oxidative dyeing is widely used because of the high color uptake as compared with direct dyeing using so-called direct dyes.

In order to perform oxidative dyeing, typically, a composition comprising oxidative base(s), as well as coupler(s), with alkaline agent(s), is mixed with a developer composition comprising oxidizing agent(s) to prepare a ready-to-use composition, and then, the ready-to-use composition is applied onto keratin fibers.

The developer composition is capable of bleaching keratin fibers due to the function of the oxidizing agent(s) in the composition. Therefore, the developer composition as well as the ready-to-use composition (this may not include any oxidation base with or without any coupler) may be used to bleach keratin fibers.

Recently, hair dyeing or bleaching compositions comprising a relatively higher amount of fatty material(s) have been proposed. For example, US-A-2010/0154140 discloses such a composition comprising mineral oil derived from petroleum.

### DISCLOSURE OF INVENTION

There is a need for a composition which can bleach or dye keratin fibers such as hair, wherein the composition can provide the keratin fibers with good bleaching or dyeing effects, while it can be friendly to environments.

Thus, an objective of the present invention is to provide a composition for keratin fibers, in particular for bleaching or dyeing of keratin fibers, which can provide the keratin fibers with good bleaching or dyeing effects, while it can be friendly to environments.

The above objective can be achieved by a composition for keratin fibers, comprising:
(a) at least one biodegradable liquid fatty material;
(b) at least one alkaline agent;
(c) at least one thickener; and
(d) water,
wherein
the amount of the (a) biodegradable liquid fatty material(s) is 10% by weight or more, preferably 30% by weight or more, and more preferably 50% by weight or more, relative to the total weight of the composition.

The (a) biodegradable liquid fatty material may be selected from oils derived from plants, ester oils, and mixtures thereof.

The oils derived from plants may comprise hydrocarbon oils, preferably alkanes, and more preferably C15-19 alkane.

The amount of the (a) biodegradable liquid fatty material(s) in the composition according to the present invention may be from 10% to 80% by weight, preferably from 30% to 75% by weight, and more preferably from 50% to 70% by weight, relative to the total weight of the composition.

The (b) alkaline agent may be selected from ammonia, alkanolamines, derivatives thereof, and salts thereof.

The alkanolamine may be selected from the group consisting of monoethanolamine, diethanolamine, triethanolamine, monoisopropanolamine, diisopropanolamine, N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, and a mixture thereof.

The amount of the (b) alkaline agent(s) in the composition according to the present invention may be from 0.1% to 20% by weight, preferably from 0.5% to 15% by weight, and more preferably from 1% to 10% by weight, relative to the total weight of the composition.

The (c) thickener may be selected from polysaccharides.

The (c) thickener may be selected from cellulose derivatives, modified guar gums, and mixtures thereof, more preferably hydroxyalkyl celluloses, guar gums modified with C₁-C₆ hydroxyalkyl groups, and mixtures thereof, and even more preferably hydroxyethylcellulose, hydroxypropyl guar, and mixtures thereof.

The amount of the (c) thickener(s) in the composition according to the present invention may be from 0.01% to 10% by weight, preferably from 0.05% to 5% by weight, and more preferably from 0.1% to 1% by weight, relative to the total weight of the composition.

The amount of the (d) water in the composition according to the present invention may be from 10% to 50% by weight, preferably from 15% to 45% by weight, and more preferably from 20% to 40% by weight, relative to the total weight of the composition.

The composition according to the present invention may further comprise (e) at least one non-ionic surfactant, preferably at least one liquid non-ionic surfactant, and more preferably selected from liquid alkyl polyglucosides.

The amount of the (e) non-ionic surfactant(s) in the composition according to the present invention may be from 0.1% to 15% by weight, preferably from 0.5% to 10% by weight, and more preferably from 1% to 5% by weight or more, relative to the total weight of the composition.

The composition according to the present invention may further comprise (f) at least one dye.

The present invention also relates to a process for keratin fibers comprising the steps of:
(1) mixing a first composition and a second composition to prepare a mixture,
   wherein
   the first composition comprises
   (a) at least one biodegradable liquid fatty material,
   (b) at least one alkaline agent,
   (c) at least one thickener, and
   (d) water, and optionally (e) at least one non-ionic surfactant and/or (f) at least one dye,
   wherein
   the amount of the (a) biodegradable liquid fatty material(s) is 10% by weight or more, preferably 30% by weight or more, and more preferably 50% by weight or more, relative to the total weight of the first composition,
   and
   the second composition comprises
   (g) at least one oxidizing agent;
and
(2) applying the mixture to the keratin fibers.

### BEST MODE FOR CARRYING OUT THE INVENTION

After diligent research, the inventors have discovered that it is possible to provide a composition for keratin fibers, in particular for bleaching or dyeing of keratin fibers, which can provide the keratin fibers with good bleaching or dyeing effects, while it can be friendly to environments.

Thus, the composition according to the present invention comprises:
(a) at least one biodegradable liquid fatty material;
(b) at least one alkaline agent;
(c) at least one thickener; and
(d) water,
wherein
the amount of the (a) biodegradable liquid fatty material(s) is 10% by weight or more, preferably 30% by weight or more, and more preferably 50% by weight or more, relative to the total weight of the composition.

The composition according to the present invention can be used as a cosmetic composition for keratin fibers such as hair, preferably a cosmetic composition for bleaching or dyeing of keratin fibers, and more preferably a cosmetic composition for bleaching or oxidative dyeing of hair.

The composition according to the present invention can provide keratin fibers with good bleaching or dyeing effects, which are at least comparable with those provided by conventional compositions for bleaching or dyeing keratin fibers.

The composition according to the present invention are friendly to environments, because it uses biodegradable fatty material. Thus, the composition according to the present invention can be decomposed in environments by the action of, for example, microorganisms, after being used for bleaching or dyeing keratin fibers.

It is preferable that the thickener be also biodegradable. It is also preferable that the non-ionic surfactant which may be added to the composition according to the present invention be biodegradable.

In addition, the present invention can reduce odor if ammonia is not used as the alkaline agent. In general, ammonia is used as an alkaline agent. However, ammonia can cause a strong odor. Therefore, it is also possible for the present invention to prevent or reduce the odor, if ammonia is not used as the alkaline agent.

Furthermore, since the biodegradable fatty material used for the composition according to the present invention is liquid, it is not necessary to heat the biodegradable fatty material to melt when preparing the composition. Therefore, the process for preparing the composition according to the present invention does not require much energy, and therefore, it is also friendly to environments.

The present invention also relates to a process for keratin fibers comprising the steps of:
(1) mixing a first composition and a second composition to prepare a mixture,
   wherein
   the first composition comprises
   (a) at least one biodegradable liquid fatty material,
   (b) at least one alkaline agent,
   (c) at least one thickener, and
   (d) water, and optionally (e) at least one non-ionic surfactant and/or (f) at least one dye,
   wherein
   the amount of the (a) biodegradable liquid fatty material(s) is 10% by weight or more, preferably 30% by weight or more, and more preferably 50% by weight or more, relative to the total weight of the first composition,
   and
   the second composition comprises
   (g) at least one oxidizing agent;
and
(2) applying the mixture to the keratin fibers.

Hereafter, the composition and process according to the present invention will be described in a detailed manner.

### [Composition]

The composition according to the present invention is intended for treating keratin fibers, and comprises:
(a) at least one biodegradable liquid fatty material;
(b) at least one alkaline agent;
(c) at least one thickener; and
(d) water,
wherein
the amount of the (a) biodegradable liquid fatty material(s) is 10% by weight or more, preferably 30% by weight or more, and more preferably 50% by weight or more, relative to the total weight of the composition.

### (Fatty Material)

The composition according to the present invention comprises (a) at least one biodegradable liquid fatty material. Two or more biodegradable liquid fatty materials may be used in combination. Thus, a single type of biodegradable liquid fatty material or a combination of different types of biodegradable liquid fatty material may be used.

The term “fatty material” means an organic compound that is insoluble in water at ambient temperature (25° C.) and at atmospheric pressure (760 mmHg) (solubility of less than 5% by weight, preferably less than 1% by weight, and even more preferentially less than 0.1% by weight).

The fatty material may contain, in its structure, at least one hydrocarbon-based chain containing at least 6 carbon atoms. In addition, the fatty material may be soluble in organic solvents under the same temperature and pressure conditions, for instance chloroform, ethanol, benzene or decamethylcyclopentasiloxane.

The fatty material used for the present invention is biodegradable. Thus, a biodegradable fatty material is used for the present invention.

The term “biodegradable” here means that the fatty material can be degraded or decomposed by any living thing, such as microorganisms, which is present in environments such as soils, air, river and sea.

It is preferable that the biodegradable fatty material has a biodegradability of more than 40%, more preferably more than 60%, and even more preferably more than 70%, based on OECD Guidelines for the Testing of Chemicals, Section 3, developed by the Organization for Economic Cooperation and Development (OECD).

The fatty material used for the present invention is liquid.

The term “liquid” here means having fluidity at ambient temperature (25° C.) under atmospheric pressure (760 mmHg). The fatty material in the form of a liquid at ambient temperature under atmospheric pressure may be referred to as “oil”.

The composition according to the present invention can have an oil phase or oil phases.

The (a) biodegradable liquid fatty material may be selected from polar oils, non-polar oils, and mixtures thereof.

The (a) biodegradable liquid fatty materials may be volatile or non-volatile.

The (a) biodegradable liquid fatty material may be selected from the group consisting of oils of animal or plant origin, synthetic glycerides, esters of fatty alcohols and/or fatty acids (fatty esters) other than animal or plant oils and synthetic glycerides, fatty acids, fatty alcohols, and hydrocarbons.

As examples of plant oils, mention may be made of, for example, linseed oil, camellia oil, macadamia nut oil, sunflower oil, apricot oil, soybean oil, arara oil, hazelnut oil, corn oil, mink oil, olive oil, avocado oil, sasanqua oil, castor oil, safflower oil, jojoba oil, sunflower (seed) oil, almond oil, grapeseed oil, sesame oil, soybean oil, peanut oil, and mixtures thereof.

As examples of animal oils, mention may be made of, for example, squalene, perhydrosqualene and squalane.

As examples of synthetic glycerides, mention may be made of, for instance, caprylic/capric acid triglycerides, for instance those sold by the company, Stearineries Dubois or those sold under the names Miglyol® 810, 812 and 818 by the company, Dynamit Nobel.

As examples of esters of fatty alcohols and/or fatty acids, which are different from the animal or plant oils as well as the synthetic glycerides mentioned above, mention may be made especially of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

The following may especially be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the fatty esters mentioned above, it is preferable to use ethyl, isopropyl, myristyl, cetyl or stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The composition may also comprise, as the fatty esters, sugar esters and diesters of C₆-C₃₀ and preferably C₁₂-C₂₂ fatty acids. The term “sugar” means oxygen-bearing hydrocarbon-based compounds containing several alcohol functions, with or without aldehyde or ketone functions, and which contain at least 4 carbon atoms. These sugars may be monosaccharides, oligosaccharides or polysaccharides.

Examples of suitable sugars that may be mentioned include sucrose (or saccharose), glucose, galactose, ribose, fructose, maltose, mannose, arabinose, xylose and lactose, and derivatives thereof, especially alkyl derivatives, such as methyl derivatives, for instance methylglucose.

The sugar esters of fatty acids may be selected especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or non-conjugated carbon-carbon double bonds.

The esters according to this variant may also be selected from mono-, di-, tri-, tetraesters and polyesters, and mixtures thereof.

These esters may be selected, for example, from oleates, laurates, palmitates, myristates, behenates, cocoates, stearates, linoleates, linolenates, caprates and arachidonates, or mixtures thereof such as, especially, oleo-palmitate, oleo-stearate and palmito-stearate mixed esters.

It is more particularly preferable to use monoesters and diesters and especially sucrose, glucose or methylglucose mono- or dioleates, stearates, behenates, oleopalmitates, linoleates, linolenates and oleostearates.

An example that may be mentioned is the product sold under the name Glucate® DO, which is a methylglucose dioleate, by the company Amerchol.

Examples of esters or mixtures of esters of sugar and of fatty acid that may also be mentioned include:
the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referred to as B370 and corresponding to sucrose behenate formed from 20% monoester and 80% di-triester-polyester;
the sucrose mono-dipalmito-stearate sold by the company Goldschmidt under the name Tegosoft® PSE.

The (a) biodegradable liquid fatty material may be at least one fatty acid, and two or more fatty acids may be used.

The fatty acids should be in acidic form (i.e., unsalified, to avoid soaps) and may be saturated or unsaturated and contain from 6 to 30 carbon atoms and in particular from 9 to 30 carbon atoms, which are optionally substituted, in particular with one or more hydroxyl groups (in particular 1 to 4). If they are unsaturated, these compounds may comprise one to three conjugated or non-conjugated carbon-carbon double bonds. They may be more particularly selected from caproic acid, caprylic acid, oleic acid, linoleic acid, linolenic acid and isostearic acid.

The (a) biodegradable liquid fatty material may be at least one fatty alcohol, and two or more fatty alcohols may be used.

The term “fatty alcohol” here means any saturated or unsaturated, linear or branched C₈-C₃₀ fatty alcohol, which is optionally substituted, in particular with one or more hydroxyl groups (in particular 1 to 4). If they are unsaturated, these compounds may comprise one to three conjugated or non-conjugated carbon-carbon double bonds.

Among the C₈-C₃₀ fatty alcohols, C₁₂-C₂₂ fatty alcohols, for example, are used. Mention may be made among these of lauryl alcohol, isostearyl alcohol, oleyl alcohol, and mixtures thereof.

As examples of hydrocarbons, mention may be made of, for example, linear or branched hydrocarbons, preferably aliphatic hydrocarbons, and more preferably alkanes. As examples of alkanes, mention may also be made of linear or branched, or possibly cyclic C₆-C₁₆ lower alkanes. Examples that may be mentioned include C9-12 alkane, C13,14 alkane and C15-19 alkane.

The hydrocarbons, such as mineral oil, derived from petroleum do not corresponds to the (a) biodegradable liquid fatty material.

It is preferable that the (a) biodegradable liquid fatty material be selected from oils derived from plants, ester oils, and mixtures thereof. The “ester oils” comprise synthetic glycerides, fatty esters, which are explained above, and mixtures thereof. The oils derived from plants may comprise hydrocarbon oils, preferably alkanes, and more preferably C15-19 alkane.

The amount of the (a) biodegradable liquid fatty material(s) in the composition according to the present invention may be 10% by weight or more, preferably 30% by weight or more, more preferably 50% by weight or more, relative to the total weight of the composition.

Furthermore, the amount of the (a) biodegradable liquid fatty material(s) in the composition according to the present invention may be 80% by weight or less, preferably 75% by weight or less, and even more preferably 70% by weight or less, relative to the total weight of the composition.

The amount of the (a) biodegradable liquid fatty material(s) in the composition according to the present invention may be from 10% to 80% by weight, preferably from 30% to 75% by weight, and more preferably from 50% to 70% by weight, relative to the total weight of the composition.

### (Alkaline Agent)

The composition according to the present invention comprises (b) at least one alkaline agent. If two or more (b) alkaline agents are used, they may be the same or different.

The (c) alkaline agent may be selected from ammonia, alkanolamines, derivatives of alkanolamines (alkanolamine derivatives), and salts of alkanolamines or alkanolamine derivatives.

The alkanolamines have an alkane structure with at least one hydroxyl group and at least one amino group.

As the alkanolamines, mention may be made of, for example, mono-, di-, and tri-ethanolamines. The alkanolamine may be selected from the group consisting of monoethanolamine, diethanolamine, triethanolamine, monoisopropanolamine, diisopropanolamine, N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, and a mixture thereof.

The alkanolamine derivative may be selected from alkanolamines in which the hydrogen atom on the nitrogen atom, if present, of the amino group in the alkanolamines is substituted with at least one substituent.

As the substituent, mention may be made of, for example, an alkyl group, an alkenyl group, and an alkynyl group.

The alkyl group may be a linear, branched or cyclic alkyl group. The alkyl group may be a linear or branched C₁-C₆ alkyl group, preferably a C₁-C₄ alkyl group, such as a methyl group, an ethyl group, a propyl group, an i-propyl group and a butyl group. On the other hand, the alkyl group may be a cyclic C₃-C₆ alkyl group, such as a cyclopentyl group and a cyclohexyl group.

The alkenyl group may be a C₂-C₆ alkenyl group such as a vinyl group, an allyl group, a butylene group, a pentenyl group and a hexenyl group.

The alkynyl group may be a C₂-C₆ alkynyl group such as an ethynyl group, and a propanyl group.

The above substituent may be further substituted with at least one group such as a halogen atom, a nitro group, a cyano group, a hydroxyl group and an aromatic group such as a phenyl group.

The type of the salts of alkanolamines and alkanolamine derivatives is not limited. The salts may be acid salts. As acid salts, mention may be made of, for example, inorganic acid salts such as hydrochloride, sulfates, nitrates, and phosphates, and organic acid salts such as citrates, oxalates, acetates, formates, maleates, and tartrates.

The amount of the (b) alkaline agent(s) in the composition according to the present invention may be from 0.1% by weight or more, preferably 0.5% by weight or more, and more preferably from 1% by weight or more, relative to the total weight of the composition.

The amount of the (b) alkaline agent(s) in the composition according to the present invention may be from 20% by weight or less, preferably from 15% by weight or less, and more preferably from 10% by weight or less, relative to the total weight of the composition.

The amount of the (b) alkaline agent(s) in the composition according to the present invention may be from 0.1% to 20% by weight, preferably from 0.5% to 15% by weight, and more preferably from 1% to 10% by weight, relative to the total weight of the composition.

### (Thickener)

The composition according to the present invention comprises (c) at least one thickener. A single type of thickener may be used, but two or more different types of thickener may be used in combination.

It is preferable that the (c) thickener be selected from polysaccharides, because polysaccharides are biodegradable.

The polysaccharides are, for example, chosen from glucans, modified and unmodified starches (such as those derived, for example, from cereals, for instance wheat, corn, or rice, from vegetables, for instance yellow peas, and tubers, for instance potatoes or cassava), amylose, amylopectin, glycogen, dextrans, celluloses, and derivatives thereof (e.g., methylcelluloses, hydroxyalkylcelluloses, hydroxyethylcelluloses, and carboxymethylcelluloses), mannans, xylans, lignins, arabans, galactans, galacturonans, chitins, chitosans, glucuronoxylans, arabinoxylans, xyloglucans, glucomannans, pectic acids, and pectins, alginic acid and alginates, arabinogalactans, carrageenans, agars, glycosaminoglucans, gum arabics, gum tragacanths, ghatti gums, karaya gums, carob gums, galactomannans, such as guar gums, and non-ionic derivatives thereof (e.g., hydroxypropyl guar), sclerotium gum and xanthan gums, and mixtures thereof.

For example, the polysaccharides that may be used are chosen from those described, for example, in “Encyclopedia of Chemical Technology”, Kirk-Othmer, Third Edition, 1982, Volume 3, pp. 896-900, and Volume 15, pp. 439-458, in “Polymers in Nature” by E. A. MacGregor and C. T. Greenwood, published by John Wiley & Sons, Chapter 6, pp. 240-328, 1980, and in “Industrial Gums-Polysaccharides and their Derivatives”, edited by Roy L. Whistler, Second Edition, published by Academic Press Inc., the content of these three publications being entirely incorporated by reference.

For example, starches, guar gums, celluloses, and derivatives thereof can be used.

Among the starches that may be used, mention may be made, for example, of macromolecules in the form of polymers comprising base units which are anhydroglucose units. The number of these units and their assembly make it possible to distinguish between amylose (linear polymer) and amylopectin (branched polymer). The relative proportions of amylose and amylopectin, as well as their degree of polymerization, can vary according to the botanical origin of the starches.

The molecules of starches used may have cereals or tubers as their botanical origin. Thus, the starches can be, for example, chosen from maize, rice, cassava, tapioca, barley, potato, wheat, sorghum, and pea starches.

Starches generally exist in the form of a white powder, insoluble in cold water, whose elementary particle size ranges from 3 to 100 microns.

The starches may be optionally C₁-C₆ hydroxyalkylated or C₁-C₆ acylated (such as acetylated). The starches may have also undergone heat treatments.

Distarch phosphates or compounds rich in distarch phosphate, such as the product provided under the references PREJEL VA-70-T AGGL (gelatinized hydroxypropylated cassava distarch phosphate) or PREJEL TK1 (gelatinized cassava distarch phosphate) or PREJEL 200 (gelatinized acetylated cassava distarch phosphate) by the company AVEBE, may also be used.

The guar gums can be modified or unmodified.

The unmodified guar gums are, for example, the products sold under the name Vidogum GH 175 by the company Unipectine and under the names Meypro-Guar 50 and Jaguar C by the company Meyhall.

The modified non-ionic guar gums are, for example, modified with C₁-C₆ hydroxyalkyl groups.

Among the hydroxyalkyl groups that may be mentioned, for example, are hydroxymethyl, hydroxyethyl, hydroxypropyl, and hydroxybutyl groups.

These guar gums are well known in the prior art and can be prepared, for example, by reacting the corresponding alkene oxides such as propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum, may, for example, range from 0.4 to 1.2.

Such non-ionic guar gums optionally modified with hydroxyalkyl groups are sold, for example, under the trade names Jaguar HP8, Jaguar HP60, Jaguar HP120, Jaguar DC 293, and Jaguar HP 105 by the company Rhodia Chimie (Meyhall) or under the name Galactasol 4H_{4FD2} by the company Aqualon.

Among the celluloses and cellulose derivatives, such as cellulose modified with hydroxylalkyl groups, that are used are, for example, hydroxypropylmethylcellulose, hydroxyethylcellulose, and hydroxypropylcellulose, as well as hydrophobicized hydroxypropylmethylcellulose. Mention may be made of the products sold under the names Klucel E F, Klucel H, Klucel L H F, Klucel M F, and Klucel G by the company Aqualon.

It is preferable that the (c) thickener be selected from hydrophilic thickeners. The hydrophilic thickeners can thicken an aqueous phase formed by the (d) water in the composition 50 according to the present invention.

It is preferable that the (c) thickener be selected from cellulose derivatives, modified guar gums, and mixtures thereof, more preferably hydroxyalkyl celluloses, guar gums modified with C₁-C₆ hydroxyalkyl groups, and mixtures thereof, and even more preferably hydroxyethylcellulose, hydroxypropyl guar, and mixtures thereof.

The amount of the (c) thickener(s) in the composition according to the present invention may be 0.01% by weight or more, preferably 0.05% by weight or more, and more preferably 0.1% by weight or more, relative to the total weight of the composition.

The amount of the (c) thickener(s) in the composition according to the present invention may be 10% by weight or less, preferably 5% by weight or less, and more preferably 1% by weight or less, relative to the total weight of the composition.

The amount of the (c) thickener(s) in the composition according to the present invention may be from 0.01% to 10% by weight, preferably from 0.05% to 5% by weight, and more preferably from 0.1% to 1% by weight, relative to the total weight of the composition.

### (Water)

The composition according to the present invention comprises (d) water.

The (d) water can form an aqueous phase of the composition according to the present invention while the (a) biodegradable liquid fatty material may form an oil phase.

If the composition according to the present invention is in the form of a W/O or O/W emulsion, the aqueous phase of the composition according to the present invention can be dispersed or inner phases in the W/O emulsion or a continuous or outer phase in the O/W emulsion.

The amount of the (d) water in the composition according to the present invention may be 10% by weight or more, preferably 15% by weight or more, and more preferably 20% by weight or more, relative to the total weight of the composition.

On the other hand, the amount of the (d) water in the composition according to the present invention may be 50% by weight or less, preferably 45% by weight or less, and more preferably 40% by weight or less, relative to the total weight of the composition.

The amount of the (d) water may be from 10% to 50% by weight, preferably from 15% to 45% by weight, and more preferably from 20% to 40% by weight, relative to the total weight of the composition.

The pH of the composition according to the present invention may be more than 7, preferably 7.5 or more, and more preferably 8 or more. Thus, the composition according to the present invention is alkaline. It is preferable that the pH of the composition according to the present invention be 13 or less, more preferably 12 or less, and even more preferably 11 or less.

The pH may be adjusted to the desired value using the (b) alkaline agent(s) and/or at least one acidifying agent.

The acidifying agents can be, for example, mineral or organic acids, for instance hydrochloric acid, phosphoric acid, carboxylic acids, for instance tartaric acid, citric acid, and lactic acid, or sulphonic acids, or ascorbic acid.

The acidifying agent may be present in an amount ranging from less than 1% by weight, preferably from 0.5% by weight or less, and more preferably 0.3% by weight or less, relative to the total weight of the composition.

### (Non-Ionic Surfactant)

The composition according to the present invention may comprise (e) at least one non-ionic surfactant. A single type of non-ionic surfactant may be used, but two or more different types of non-ionic surfactants may be used in combination.

The non-ionic surfactant may have an HLB (Hydrophilic Lipophilic Balance) value of from 3.0 to 7.0, preferably from 3.5 to 6.0, and more preferably from 4.0 to 5.0. Alternatively, the non-ionic surfactant may have an HLB value of from 11 to 17, preferably from 12 to 16, and more preferably from 13 to 15. If two or more non-ionic surfactants are used, the HLB value is determined by the weighted average of the HLB values of all the non-ionic surfactants.

The non-ionic surfactant may be chosen from:
(1) surfactants chosen from polyglyceryl fatty acid esters, polyoxyalkylenated alkyl glycerides, and polyoxyalkylenated fatty ethers;
(2) mixed esters of fatty acid or of fatty alcohol, of carboxylic acid and of glycerol;
(3) fatty acid esters of sugars and fatty alcohol ethers of sugars;
(4) surfactants chosen from fatty esters of sorbitan and oxyalkylenated fatty esters of sorbitan, and oxyalkylenated fatty esters;
(5) block copolymers of ethylene oxide (A) and of propylene oxide (B),
(6) polyoxyethylenated (1-40 EO) and polyoxypropylenated (1-30 PO) alkyl (C₁₆-C₃₀) ethers,
(7) silicone surfactants, and
(8) mixtures thereof.

The surfactant (1) may be a fluid at a temperature of less than or equal to 45° C.

The surfactant (1) may be in particular:
polyglyceryl fatty acid esters of at least one, preferably one, fatty acid comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2-12 glycerols, preferably 2-10 glycerols and more preferably 2-8 glycerols;
polyoxyethylenated (PEGylated) alkyl glycerides such as polyethylene glycol derivatives of a mixture of mono-, di- and tri-glycerides of caprylic and capric acids (preferably 2 to 30 ethylene oxide units, more preferably 2 to 20 ethylene oxide units, and even more preferably 2 to 10 ethylene oxide units), e.g., PEG-6 Caprylic/Capric Glycerides, PEG-7 Caprylic/Capric Glycerides, and PEG-7 glyceryl cocoate;
polyoxyethylenated fatty ethers of at least one, preferably one, fatty alcohol comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2 to 60 ethylene oxides, preferably from 2 to 30 ethylene oxides, and more preferably from 2 to 10 ethylene oxides; and
mixtures thereof.

It is preferable that the polyglyceryl fatty acid ester have a polyglycerol moiety derived from 2 to 10 glycerols, more preferably from 2 to 8 glycerols, and further more preferably 4 to 6 glycerols.

The polyglyceryl fatty acid ester may be chosen from the mono, di and tri esters of saturated or unsaturated acid, preferably saturated acid, including 8 to 22 carbon atoms, preferably 8 to 18 carbon atoms, and more preferably 8 to 12 carbon atoms, such as caprylic acid, capric acid, lauric acid, oleic acid, stearic acid, isostearic acid, and myristic acid.

The polyglyceryl fatty acid ester may be selected from the group consisting of PG2 caprate, PG2 dicaprate, PG2 tricaprate, PG2 caprylate, PG2 dicaprylate, PG2 tricaprylate, PG2 laurate, PG2 dilaurate, PG2 trilaurate, PG2 myristate, PG2 dimyristate, PG2 trimyristate, PG2 stearate, PG2 distearate, PG2 tristearate, PG2 isostearate, PG2 diisostearate, PG2 triisostearate, PG2 oleate, PG2 dioleate, PG2 trioleate, PG3 caprate, PG3 dicaprate, PG3 tricaprate, PG3 caprylate, PG3 dicaprylate, PG3 tricaprylate, PG3 laurate, PG3 dilaurate, PG3 trilaurate, PG3 myristate, PG3 dimyristate, PG3 trimyristate, PG3 stearate, PG3 distearate, PG3 tristearate, PG3 isostearate, PG3 diisostearate, PG3 triisostearate, PG3 oleate, PG3 dioleate, PG3 trioleate, PG4 caprate, PG4 dicaprate, PG4 tricaprate, PG4 caprylate, PG4 dicaprylate, PG4 tricaprylate, PG4 laurate, PG4 dilaurate, PG4 trilaurate, PG4 myristate, PG4 dimyristate, PG4 trimyristate, PG4 stearate, PG4 distearate, PG4 tristearate, PG4 isostearate, PG4 diisostearate, PG4 triisostearate, PG4 oleate, PG4 dioleate, PG4 trioleate, PG5 caprate, PG5 dicaprate, PG5 tricaprate, PG5 caprylate, PG5 dicaprylate, PG5 tricaprylate, PG5 laurate, PG5 dilaurate, PG5 trilaurate, PG5 myristate, PG5 dimyristate, PG5 trimyristate, PG5 stearate, PG5 distearate, PG5 tristearate, PG5 isostearate, PG5 diisostearate, PG5 triisostearate, PG5 oleate, PG5 dioleate, PG5 trioleate, PG6 caprate, PG6 dicaprate, PG6 tricaprate, PG6 caprylate, PG6 dicaprylate, PG6 tricaprylate, PG6 laurate, PG6 dilaurate, PG6 trilaurate, PG6 myristate, PG6 dimyristate, PG6 trimyristate, PG6 stearate, PG6 distearate, PG6 tristearate, PG6 isostearate, PG6 diisostearate, PG6 triisostearate, PG6 oleate, PG6 dioleate, PG6 trioleate, PG10 caprate, PG10 dicaprate, PG10 tricaprate, PG10 caprylate, PG10 dicaprylate, PG10 tricaprylate, PG10 laurate, PG10 dilaurate, PG10 trilaurate, PG10 myristate, PG10 dimyristate, PG10 trimyristate, PG10 stearate, PG10 distearate, PG10 tristearate, PG10 isostearate, PG10 diisostearate, PG10 triisostearate, PG10 oleate, PG10 dioleate, and PG10 trioleate.

The polyoxyalkylenated fatty ethers, preferably polyoxyethylenated fatty ethers, may comprise from 2 to 60 ethylene oxide units, preferably from 2 to 30 ethylene oxide units, and more preferably from 2 to 10 ethylene oxide units. The fatty chain of the ethers may be chosen in particular from lauryl, behenyl, arachidyl, stearyl and cetyl units, and mixtures thereof, such as cetearyl. Examples of ethoxylated fatty ethers which may be mentioned are lauryl alcohol ethers comprising 2, 3, 4, and 5 ethylene oxide units (CTFA names: Laureth-2, Laureth-3, Laureth-4, and Laureth-5), such as the products sold under the names Nikkol BL-2 by the company Nikko Chemicals, Emalex 703 by the company Nihon Emulsion Co., Ltd, Nikkol BL-4 by the company Nikko Chemicals, and EMALEX 705 by the company Nihon Emulsion Co., Ltd. Mention may also be made of, for example, stearyl alcohol ethers comprising 2, 3, 4 and 5 ethylene oxide units (CTFA names: Steareth-2, Steareth-3, Steareth-4, and Steareth-5), such as the products sold under the names Emalex 602 by the company Nihon Emulsion Co., Ltd., Emalex 603 by the company Nihon Emulsion Co., Ltd, Nikkol BS-4 by the company Nikko Chemicals, and Emalex 605 by the company Nihon Emulsion Co., Ltd.

The (2) mixed esters of fatty acids, or of fatty alcohol, of carboxylic acid and of glycerol, which can be used as the above non-ionic surfactant, may be chosen in particular from the group comprising mixed esters of fatty acid or of fatty alcohol with an alkyl or alkenyl chain containing from 8 to 22 carbon atoms, preferably from 8 to 18 carbon atoms, and more preferably from 8 to 12 carbon atoms, and of α-hydroxy acid and/or of succinic acid, with glycerol. The α-hydroxy acid may be, for example, citric acid, lactic acid, glycolic acid or malic acid, and mixtures thereof.

The alkyl chain of the fatty acids or alcohols from which are derived the mixed esters which can be used in the nanoemulsion of the present invention may be linear or branched, and saturated or unsaturated. They may especially be stearate, isostearate, linoleate, oleate, behenate, arachidonate, palmitate, myristate, laurate, caprate, isostearyl, stearyl, linoleyl, oleyl, behenyl, myristyl, lauryl or capryl chains, and mixtures thereof.

As examples of mixed esters which can be used in the nanoemulsion of the present invention, mention may be made of the mixed ester of glycerol and of the mixture of citric acid, lactic acid, linoleic acid and oleic acid (CTFA name: Glyceryl citrate/lactate/linoleate/oleate) sold by the company Huls under the name Imwitor 375; the mixed ester of succinic acid and of isostearyl alcohol with glycerol (CTFA name: Isostearyl diglyceryl succinate) sold by the company Hüls under the name Imwitor 780 K; the mixed ester of citric acid and of stearic acid with glycerol (CTFA name: Glyceryl stearate citrate) sold by the company Huls under the name Imwitor 370; the mixed ester of lactic acid and of stearic acid with glycerol (CTFA name: Glyceryl stearate lactate) sold by the company Danisco under the name Lactodan B30 or Rylo LA30.

The (3) fatty acid esters of sugars, which can be used as the above non-ionic surfactant, may be chosen in particular from the group comprising esters or mixtures of esters of C₈-C₂₂ fatty acid and of sucrose, of maltose, of glucose or of fructose, and esters or mixtures of esters of C₁₄-C₂₂ fatty acid and of methylglucose.

The C₈-C₂₂ or C₁₄-C₂₂ fatty acids forming the fatty unit of the esters which can be used in the present invention comprise a saturated or unsaturated linear alkyl or alkenyl chain containing, respectively, from 8 to 22 or from 14 to 22 carbon atoms. The fatty unit of the esters may be chosen in particular from stearates, behenates, arachidonates, palmitates, myristates, laurates and caprates, and mixtures thereof. Stearates are preferably used.

As examples of esters or mixtures of esters of fatty acid and of sucrose, of maltose, of glucose or of fructose, mention may be made of sucrose monostearate, sucrose distearate and sucrose tristearate and mixtures thereof, such as the products sold by the company Croda under the name Crodesta F50, F70, F110 and F160; and examples of esters or mixtures of esters of fatty acid and of methylglucose which may be mentioned are methylglucose polyglyceryl-3 distearate, sold by the company Goldschmidt under the name Tego-care 450. Mention may also be made of glucose or maltose monoesters such as methyl o-hexadecanoyl-6-D-glucoside and o-hexadecanoyl-6-D-maltoside.

The (3) fatty alcohol ethers of sugars, which can be used as the above non-ionic surfactant, may be solid at a temperature of less than or equal to 45° C. and may be chosen in particular from the group comprising ethers or mixtures of ethers of C₈-C₂₂ fatty alcohol and of glucose, of maltose, of sucrose or of fructose, and ethers or mixtures of ethers of a C₁₄-C₂₂ fatty alcohol and of methylglucose. These are in particular alkylpolyglucosides.

The C₈-C₂₂ or C₁₄-C₂₂ fatty alcohols forming the fatty unit of the ethers which may be used in the nanoemulsion of the present invention comprise a saturated or unsaturated, linear alkyl or alkenyl chain containing, respectively, from 8 to 22 or from 14 to 22 carbon atoms. The fatty unit of the ethers may be chosen in particular from decyl, cetyl, behenyl, arachidyl, stearyl, palmityl, myristyl, lauryl, capryl and hexadecanoyl units, and mixtures thereof, such as cetearyl.

As examples of fatty alcohol ethers of sugars, mention may be made of alkylpolyglucosides such as decylglucoside and laurylglucoside, which is sold, for example, by the company Henkel under the respective names Plantaren 2000 and Plantaren 1200, cetostearyl glucoside optionally as a mixture with cetostearyl alcohol, sold for example, under the name Montanov 68 by the company SEPPIC, under the name Tego-care CG90 by the company Goldschmidt and under the name Emulgade KE3302 by the company Henkel, as well as arachidyl glucoside, for example in the form of a mixture of arachidyl alcohol and behenyl alcohol and arachidyl glucoside, sold under the name Montanov 202 by the company SEPPIC.

The surfactant used more particularly is sucrose monostearate, sucrose distearate or sucrose tristearate and mixtures thereof, methylglucose polyglyceryl-3 distearate and alkylpolyglucosides.

As alkylpolyglucosides, those containing an alkyl group including from 6 to 30 carbon atoms, and preferably from 8 to 16 carbon atoms, and containing a hydrophilic group (glucoside) preferably comprising 1.2 to 3 saccharide units, are preferably used. Mention may for example be made of decylglucoside (Alkyl-C9/C11-polyglucoside (1.4)) like the product marketed under the name of MYDOL 10® by Kao Chemicals, the product marketed under the name of PLANTAREN 2000 UP® by Cognis, and the product marketed under the name of ORAMIX NS 10® by Seppic; caprylyl/capryl glucoside like the product marketed under the name of ORAMIX CG 110® by Seppic or PLANTACARE 810 P by Cognis; laurylglucoside like the products marketed under the names of PLANTAREN 1200 N® and PLANTACARE 1200® by Cognis; and coco-glucoside like the product marketed under the name of PLANTACARE 818/UP® by Cognis, cetostearyl glucoside optionally mixed with cetostearylic alcohol, for example marketed under the name of MONTANOV 68 by Seppic, under the name of TEGO-CARE CG90 by Goldschmidt and under the name of EMULGADE KE3302 by Henkel; arachidyl glucoside, for example in the form of a mixture of arachidic and behenic alcohols and arachidyl glucoside marketed under the name of MONTANOV 202 by Seppic; cocoylethylglucoside, for example in the form of the mixture (35/65) with cetyl and stearyl alcohols, marketed under the name of MONTANOV 82 by Seppic, C12-C20 alkyl glucosides such as those marketed as a mixture with C14-C22 fatty alcohols under the reference of MONTANOV L by Seppic.

The (4) fatty esters of sorbitan and oxyalkylenated fatty esters of sorbitan which may be used as the above non-ionic surfactant may be chosen from the group comprising C₁₆-C₂₂ fatty acid esters of sorbitan and oxyethylenated C₁₆-C₂₂ fatty acid esters of sorbitan. They may be formed from at least one fatty acid comprising at least one saturated linear alkyl chain containing, respectively, from 16 to 22 carbon atoms, and from sorbitol or from ethoxylated sorbitol. The oxyethylenated esters may generally comprise from 1 to 100 ethylene glycol units and preferably from 2 to 40 ethylene oxide (EO) units.

These esters may be chosen in particular from stearates, behenates, arachidates, palmitates, and mixtures thereof. Stearates and palmitates are preferably used.

As examples of the above non-ionic surfactant which can be used in the present invention, mention may be made of sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65.

The (4) oxyalkylenated fatty esters, preferably ethoxylated fatty esters, which may be used as the above non-ionic surfactant, may be esters formed from 1 to 100 ethylene oxide units, preferably from 2 to 60 ethylene oxide units, and more preferably from 2 to 30 ethylene oxide units, and from at least one fatty acid chain containing from 8 to 22 carbon atoms, preferably from 8 to 18 carbon atoms, and more preferably from 8 to 12 carbon atoms. The fatty chain in the esters may be chosen in particular from stearate, behenate, arachidate and palmitate units, and mixtures thereof. Examples of ethoxylated fatty esters which may be mentioned are the ester of stearic acid comprising 40 ethylene oxide units, such as the product sold under the name Myrj 52 (CTFA name: PEG-40 stearate) by the company ICI, as well as the ester of behenic acid comprising 8 ethylene oxide units (CTFA name: PEG-8 behenate), such as the product sold under the name Compritol HD5 ATO by the company Gattefosse.

The (5) block copolymers of ethylene oxide (A) and of propylene oxide (B), which may be used as the above non-ionic surfactant, may be chosen in particular from block copolymers of formula (I):

HO(C₂H₄O)ₓ(C₃H₆O)_{y}(C₂H₄O)_{z}H (I)

in which x, y and z are integers such that x+z ranges from 2 to 100 and y ranges from 14 to 60, and mixtures thereof, and more particularly from the block copolymers of formula (I) having an HLB value ranging from 8.0 to 14.0.

The (6) polyoxyethylenated (1-40 EO) and polyoxypropylenated (1-30 PO) alkyl (C₁₆-C₃₀) ethers, which may be used as the above non-ionic surfactant, may be selected from the group consisting of:
PPG-6 Decyltetradeceth-30; Polyoxyethylene (30) Polyoxypropylene (6) Tetradecyl Ether such as those sold as Nikkol PEN-4630 from Nikko Chemicals Co.,
PPG-6 Decyltetradeceth-12; Polyoxyethylene (12) Polyoxypropylene (6) Tetradecyl Ether such as those sold as Nikkol PEN-4612 from Nikko Chemicals Co.,
PPG-13 Decyltetradeceth-24; Polyoxyethylene (24) Polyoxypropylene (13) Decyltetradecyl Ether such as those sold as UNIIUBE 50MT-2200B from NOF Corporation,
PPG-6 Decyltetradeceth-20; Polyoxyethylene (20) Polyoxypropylene (6) Decyltetradecyl Ether such as those sold as Nikkol PEN-4620 from Nikko Chemicals Co.,
PPG-4 Ceteth-1; Polyoxyethylene (1) Polyoxypropylene (4) Cetyl Ether such as those sold as Nikkol PBC-31 from Nikko Chemicals Co.,
PPG-8 Ceteth-1; Polyoxyethylene (1) Polyoxypropylene (8) Cetyl Ether such as those sold as Nikkol PBC-41 from Nikko Chemicals Co.,
PPG-4 Ceteth-10; Polyoxyethylene (10) Polyoxypropylene (4) Cetyl Ether such as those sold as Nikkol PBC-33 from Nikko Chemicals Co.,
PPG-4 Ceteth-20; Polyoxyethylene (20) Polyoxypropylene (4) Cetyl Ether such as those sold as Nikkol PBC-34 from Nikko Chemicals Co.,
PPG-5 Ceteth-20; Polyoxyethylene (20) Polyoxypropylene (5) Cetyl Ether such as those sold as Procetyl AWS from Croda Inc.,
PPG-8 Ceteth-20; Polyoxyethylene (20) Polyoxypropylene (8) Cetyl Ether such as those sold as Nikkol PBC-44 from Nikko Chemicals Co., and
PPG-23 Steareth-34; Polyoxyethylene Polyoxypropylene Stearyl Ether (34 EO) (23 PO) such as those sold as Unisafe 34S-23 from Pola Chemical Industries. They can provide a composition with stability over a long time, even though the temperature of the composition is increased and decreased in a relatively short period of time.

It is more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-6 Decyltetradeceth-20, PPG-5 Ceteth-20, PPG-8 Ceteth-20, and PPG-23 Steareth-34.

It is even more preferable that the polyoxyethylenated (1-40 EO) and polyoxypropylenated (1-30 PO) alkyl (C₁₆-C₃₀) ethers are (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-5 Ceteth-20, and PPG-8 Ceteth-20.

As (7) silicone surfactants, which may be used as the above non-ionic surfactant, mention may be made of those disclosed in documents U.S. Pat. Nos. 5,364,633 and 5,411,744.

The (7) silicone surfactant as the above non-ionic surfactant may preferably be a compound of formula (I):

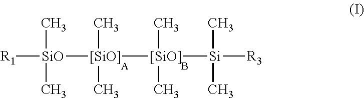

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; with the proviso that A and B are not simultaneously equal to zero;
x is an integer ranging from 1 to 6;
y is an integer ranging from 1 to 30; and
z is an integer ranging from 0 to 5.

According to one preferred embodiment of the present invention, in the compound of formula (I), the alkyl radical is a methyl radical, x is an integer ranging from 2 to 6, and y is an integer ranging from 4 to 30.

As examples of silicone surfactants of formula (I), mention may be made of the compounds of formula (II):

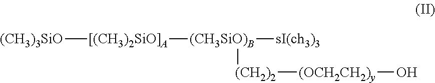

in which A is an integer ranging from 20 to 105, B is an integer ranging from 2 to 10, and y is an integer ranging from 10 to 20.

As examples of silicone surfactants of formula (I), mention may also be made of the compounds of formula (III):

H—(OCH₂CH₂)_{y}—(CH₂)₃—[(CH₃)₂SiO]_{A′}—(CH₂)₃—(OCH₂CH₂)_{y}—OH (III)
in which A′ and y are integers ranging from 10 to 20.

Compounds of the present invention which may be used are those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2, and y is 12; A is 103, B is 10, and y is 12; A is 27, B is 3, and y is 12.

The compound Q4-3667 is a compound of formula (III) in which A is 15 and y is 13.

It is preferable that the (e) non-ionic surfactant be liquid.

The term “liquid” here means having fluidity at ambient temperature (25° C.) under atmospheric pressure (760 mmHg).

It is more preferable that the liquid non-ionic surfactant be selected from liquid alkyl polyglucosides.

The amount of the (e) non-ionic surfactant(s) in the composition according to the present invention may be 0.1% by weight or more, preferably 0.5% by weight or more, and more preferably 1% by weight or more relative to the total weight of the composition.

On the other hand, the amount of the (e) non-ionic surfactant(s) in the composition according to the present invention may be 15% by weight or less, preferably 10% by weight or less, and more preferably 5% by weight or less relative to the total weight of the composition.

The amount of the (e) non-ionic surfactant(s) in the composition according to the present invention may range from 0.1% to 15% by weight, preferably from 0.5% to 10% by weight, more preferably from 1% to 5% by weight relative to the total weight of the composition.

### (Dye)

The composition according to the present invention may comprise (f) at least one dye. If two or more (f) dyes are used, they may be the same or different.

It is preferable that the dye be selected from oxidative dyes.

The oxidative dyes may be selected from oxidation bases and couplers.

The oxidation base can be selected from those conventionally known in oxidation dyeing, preferably from the group consisting of ortho- and para-phenylenediamines, double bases, ortho- and para-aminophenols, heterocyclic bases, and the acid addition salts thereof.

There may be mentioned in particular:
(I) the para-phenylenediamines of the following formula (I) and their addition salts with an acid:

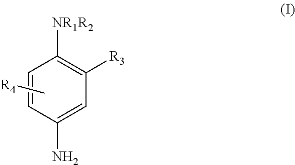

in which:
R₁ represents a hydrogen atom, a C₁-C₄ alkyl radical, a monohydroxy(C₁-C₄ alkyl) radical, a polyhydroxy-(C₂-C₄ alkyl) radical, a (C₁-C₄)alkoxy(C₁-C₄)alkyl radical, a C₁-C₄ alkyl radical substituted with a nitrogen-containing group, a phenyl radical, or a 4′-aminophenyl radical;
R₂ represents a hydrogen atom, a C₁-C₄ alkyl radical, a monohydroxy(C₁-C₄ alkyl) radical, a polyhydroxy(C₂-C₄ alkyl) radical, a (C₁-C₄)alkoxy(C₁-C₄)alkyl radical, or a C₁-C₄ alkyl radical substituted with a nitrogen-containing group;
R₁ and R₂ may also form with the nitrogen atom carrying them a 5- or 6-membered nitrogen-containing heterocycle optionally substituted with one or more alkyl, hydroxyl, or ureido groups;
R₃ represents a hydrogen atom, a halogen atom such as a chlorine atom, a C₁-C₄ alkyl radical, a sulpho radical, a carboxyl radical, a monohydroxy(C₁-C₄ alkyl) radical, a hydroxy(C₁-C₄ alkoxy) radical, an acetylamino(C₁-C₄ alkoxy) radical, a mesylamino(C₁-C₄ alkoxy) radical, or a carbamoylamino(C₁-C₄ alkoxy) radical; and
R₄ represents a hydrogen or halogen atom or a C₁-C₄ alkyl radical.

Among the nitrogen-containing groups of formula (I) above, there may be mentioned in particular the amino, mono(C₁-C₄)alkylamino, (C₁-C₄)dialkylamino, (C₁-C₄)trialkylamino, monohydroxy(C₁-C₄)alkylamino, di(monohydroxy(C₁-C₄)alkyl)amino, imidazolinium, and ammonium radicals.

Among the para-phenylenediamines of formula (I) above, mention may be more particularly made of para-phenylenediamine, para-tolylenediamine, 2-chloro-paraphenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethylpara-phenylenediamine, N,N-diethyl-para-phenylenediamine, N,N-dipropyl-paraphenylenediamine, 4-amino-N,N-diethyl-3-methylaniline, N,N-bis(3-hydroxyethyl)-paraphenylenediamine, 4-N,N-bis(3-hydroxyethyl)amino-2-methylaniline, 4-N,N-bis(3-hydroxyethyl)amino-2-chloroaniline, 2-β-hydroxyethyl-para-phenylenediamine, 2-fluoro-paraphenylenediamine, 2-isopropyl-para-phenylenediamine, N-(β-hydroxypropyl)-paraphenylenediamine, 2-hydroxymethyl-para-phenylenediamine, N,N-dimethyl-3-methylpara-phenylenediamine, N,N-(ethyl-β-hydroxyethyl)-para-phenylenediamine, N-(0,T-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylamino-ethyloxy-para-phenylenediamine, N-(β-methoxyethyl)-para-phenylenediamine, 2-methyl-1-N-β-hydroxyethyl-para-phenylenediamine, N-(4-aminophenyl)-3-hydroxy-pyrrolidine, 2-[{2-[(4-aminophenyl)amino]ethyl}(2-hydroxyethyl)amino]-ethanol, and their addition salts with an acid.

Among the para-phenylenediamines of formula (I) above, most particularly preferred are para-phenylenediamine, para-tolylenediamine, 2-isopropyl-paraphenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(3-hydroxyethyl)-para-phenylenediamine, 2-chloro-para-phenylenediamine, and their addition salts with an acid.
(II) According to the present invention, “double bases” are understood to mean compounds containing at least two aromatic rings on which amino and/or hydroxyl groups are carried.

Among the double bases which can be used as oxidation bases in the dyeing compositions in accordance with the present invention, mention may be made in particular of compounds corresponding to the following formula (II), and their addition salts with an acid:

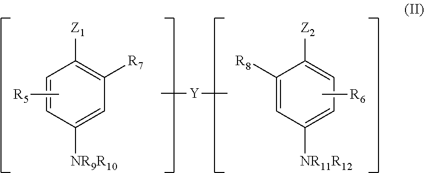

in which:
   Z₁ and Z₂, which are identical or different, represent a hydroxyl or —NH₂ radical which may be substituted with a C₁-C₄ alkyl radical or with a linking arm Y;
   the linking arm Y represents a linear or branched alkylene chain comprising from 1 to 14 carbon atoms, which may be interrupted by or which may end with one or more nitrogen-containing groups and/or one or more heteroatoms such as oxygen, sulphur, or nitrogen atoms, and optionally substituted with one or more hydroxyl or C₁-C₆ alkoxy radicals;
   R₅ and R₆ represent a hydrogen or halogen atom, a C₁-C₄ alkyl radical, a monohydroxy(C₁-C₄ alkyl) radical, a polyhydroxy(C₂-C₄ alkyl) radical, an amino(C₁-C₄ alkyl) radical, or a linking arm Y;
   R₇, R₈, R₉, R₁₀, R₁₁, and R₁₂, which are identical or different, represent a hydrogen atom, a linking arm Y, or a C₁-C₄ alkyl radical; it being understood that the compounds of formula (II) contain only one linking arm Y per molecule.

Among the nitrogen-containing groups of formula (II) above, mention may be made in particular of the amino, mono(C₁-C₄)alkylamino, (C₁-C₄)dialkylamino, (C₁-C₄)trialkylamino, monohydroxy(C₁-C₄)alkylamino, imidazolinium, and ammonium radicals.

Among the double bases of formulae (II) above, mention may be more particularly made of N,N′-bis(β-hydroxyethyl)-N,N′-bis(4′-aminophenyl)-1,3-diaminopropanol, N,N′-bis(p-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylene-diamine, 1,8-bis(2,5-diaminophenoxy)-3,5-dioxaoctane, and their addition salts with an acid.

Among these double bases of formula (II), N,N′-bis(β-hydroxyethyl)-N,N′-bis(4′-aminophenyl)-1,3-diaminopropanol, 1,8-bis(2,5-diaminophenoxy)-3,5-dioxaoctane, or one of their addition salts with an acid are particularly preferred.
(III) The para-aminophenols corresponding to the following formula (III), and their addition salts with an acid:

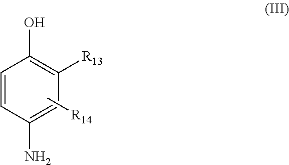

in which:
   R₃ represents a hydrogen atom, or a halogen atom such as fluorine, a C₁-C₄ alkyl, monohydroxy(C₁-C₄ alkyl), (C₁-C₄)alkoxy(C₁-C₄)-alkyl, amino(C₁-C₄ alkyl), or hydroxy(C₁-C₄)alkylamino-(C₁-C₄ alkyl) radical, and
   R₁₄ represents a hydrogen atom, or a halogen atom such as fluorine, a C₁-C₄ alkyl, monohydroxy(C₁-C₄ alkyl), polyhydroxy(C₂-C₄ alkyl), amino(C₁-C₄ alkyl), cyano(C₁-C₄ alkyl), or (C₁-C₄)alkoxy(C₁-C₄)alkyl radical.

Among the para-aminophenols of formula (III) above, mention may be more particularly made of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and their addition salts with an acid.
(IV) The ortho-aminophenols which can be used as oxidation bases in the context of the present invention are chosen in particular from 2-aminophenol, 2-amino-1-hydroxy-5-methylbenzene, 2-amino-i-hydroxy-6-methylbenzene, 5-acetamido-2-aminophenol, and their addition salts with an acid.
(V) Among the heterocyclic bases which can be used as oxidation bases in the dyeing compositions in accordance with the present invention, mention may be more particularly made of pyridine derivatives, pyrimidine derivatives, pyrazole derivatives, and their addition salts with an acid.

Among the pyridine derivatives, mention may be more particularly made of the compounds described for example in patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Among the pyrimidine derivatives, mention may be more particularly made of the compounds described, for example, in patents DE 2 359 399; JP 88-169571; and JP 91-10659, or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triamino-pyrimidine, and the pyrazolopyrimidine derivatives such as those mentioned in patent application FR-A-2 750 048 and among which there may be mentioned pyrazolo[1,5-a]-pyrimidine-3,7-diamine; 2,5-dimethyl-pyrazolo[1,5-a]-pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-amino-pyrazolo[1,5-a]pyrimidin-5-ol; 2-(3-amino-pyrazolo-[1,5-a]pyrimidin-7-ylamino)ethanol, 2-(7-aminopyrazolo[1,5-a]pyrimidin-3-ylamino)ethanol, 2-[(3-amino-pyrazolo[1,5-a]pyrimidin-7-yl)-(2-hydroxy-ethyl)amino]-ethanol, 2-[(7-aminopyrazolo[1,5-a]-pyrimidin-3-yl)-(2-hydroxyethyl)amino]ethanol, 5,6-dimethylpyrazolo-[1,5-a]pyrimidine-3,7-diamine, 2,6-dimethylpyrazolo-[1,5-a]pyrimidine-3,7-diamine, 2,5,N7,N7-tetramethyl-pyrazolo[1,5-a]pyrimidine-3,7-diamine, 3-amino-5-methyl-7-imidazolylpropyl-aminopyrazolo[1,5-a]-pyrimidine, their addition salts and their tautomeric forms, when a tautomeric equilibrium exists, and their addition salts with an acid.

Among the pyrazole derivatives, mention may more particularly be made of the compounds described in patents DE 3 843 892 and DE 4 133 957 and patent applications WO 94/08969, WO 94/08970, FR-A-2 733 749, and DE 195 43 988 such as 4,5-diamino-1-methylpyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazino-pyrazole, 1-benzyl-4,5-diamino-3-methyl-pyrazole, 4,5-diamino-3-tert-butyl-1-methylpyrazole, 4,5-diamino-1-tertbutyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxy-methylpyrazole, 4,5-diamino-3-hydroxymethyl-1-methylpyrazole, 4,5-diamino-3-hydroxymethyl-1-isopropyl-pyrazole, 4,5-diamino-3-methyl-1-isopropyl-pyrazole, 4-amino-5-(2′-aminoethyl)amino-1,3-dimethylpyrazole, 3,4,5-triaminopyrazole, 1-methyl-3,4,5-triamino-pyrazole, 3,5-diamino-1-methyl-4-methylaminopyrazole, 3,5-diamino-4-(β-hydroxy-ethyl)amino-1-methylpyrazole, and their addition salts with an acid.

Among the heterocyclic bases which can be used as oxidation bases, mention may more particularly be made of diaminopyrazolopyrazolones and especially 2,3-diamino-6,7-dihydro-1H5H-[pyrazolol,2,a]pyrazol-1-one and the addition salts of these diaminopyrazolopyrazolones with an acid.

The coupler may be an oxidation coupler which can be selected from those conventionally known in oxidation dyeing, preferably from the group consisting of meta-phenylenediamines, meta-aminophenols, meta-diphenols, naphthols, heterocyclic couplers, and the acid addition salts thereof.

The heterocyclic couplers may be selected from the group consisting of indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and their addition salts with an acid.

These couplers are more particularly chosen from 2,4-diamino-1-(3-hydroxyethyloxy)benzene, 2-methyl-5-aminophenol, 5-N-(β-hydroxyethyl)amino-2-methylphenol, 3-aminophenol, 2-chloro-3-amino-6-methylphenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 2-methyl-5-hydroxyethylaminophenol, 4-amino-2-hydroxytoluene, 1,3-bis(2,4-diaminophenoxy)-propane, sesamol, 1-amino-2-methoxy-4,5-methylene-dioxybenzene, α-naphthol, 6-hydroxyindole, 4-hydroxyindole, 4-hydroxy-N-methylindole, 6-hydroxy-indoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethyl-pyrazolo[3,2-c]-1,2,4-triazole, 2,6-dimethylpyrazolo[1,5-b]-1,2,4-triazole, and their addition salts with an acid.

In general, the addition acid salts of the oxidation bases and couplers are chosen in particular from hydrochlorides, hydrobromides, sulfates, citrates, succinates, tartrates, lactates, tosylates, benzenesulfonates, phosphates, and acetates.

The amount of the (f) dye(s) in the composition according to the present invention may be 0.1% by weight or more, preferably 0.5% by weight or more, and more preferably 1% by weight or more, relative to the total weight of the composition.

On the other hand, the amount of the (f) dye(s) in the composition according to the present invention may be 15% by weight or less, preferably 10% by weight or less, and more preferably 5% by weight or less, relative to the total weight of the composition.

The amount of the (f) dye(s) in the composition according to the present invention may be from 0.1% to 15% by weight, preferably from 0.5% to 10% by weight, and more preferably from 1% to 5% by weight, relative to the total weight of the composition.

### (Other Ingredients)

The composition according to the present invention may also include at least one optional or additional ingredient.

The amount of the optional or additional ingredient(s) is not limited, but may be from 0.01% to 30% by weight, preferably from 0.1% to 20% by weight, and more preferably from 1% to 10% by weight, relative to the total weight of the composition according to the present invention.

The optional or additional ingredient(s) may be selected from the group consisting of cationic, anionic or amphoteric surfactants; UV filters; peptides and derivatives thereof, protein hydrolyzates; swelling agents and penetrating agents; agents for combating hair loss; anti-dandruff agents; suspending agents; sequestering agents; opacifying agents; vitamins or provitamins; fragrances; preserving agents, stabilizers; and mixtures thereof.

The aqueous phase of the composition according to the present invention may include, in addition to water, one or several cosmetically acceptable organic solvents, which may be alcohols: in particular monovalent alcohols such as ethyl alcohol, isopropyl alcohol, benzyl alcohol and phenylethyl alcohol; sugars; sugar alcohols; and ethers such as ethylene glycol monomethyl, monoethyl and monobutyl ethers, propylene glycol monomethyl, monoethyl and monobutyl ether, and butylene glycol monomethyl, monoethyl and monobutyl ethers.

The organic solvent(s) may then be present in a concentration of from 0.01% to 30% by weight, preferably from 0.1% to 20% by weight, and more preferably from 1% to 15% by weight, relative to the total weight of the composition.

It is preferable that the composition according to the present invention is free from non-biodegradable or poor biodegradable ingredients such as mineral oil.

The amount of the non-biodegradable or poor biodegradable ingredients such as mineral oil in the composition according to the present invention may be 1% by weight or less, preferably 0.1% by weight or less, and more preferably 0.01% by weight or less, relative to the total weight of the composition. It is in particular preferable that the composition according to the present invention include no non-biodegradable or poor biodegradable ingredients such as mineral oil.

### (Preparation)

The composition according to the present invention can be prepared by mixing the above-described essential and optional ingredients in a conventional manner.

For example, the composition according to the present invention can be prepared by a process comprising the step of mixing
(a) at least one biodegradable liquid fatty material;
(b) at least one alkaline agent;
(c) at least one thickener; and
(d) water, and optionally (e) at least one non-ionic surfactant and/or (f) at least one dye,
wherein
the amount of the (a) biodegradable liquid fatty material(s) is 10% by weight or more, preferably 30% by weight or more, and more preferably 50% by weight or more, relative to the total weight of the composition.

For the above ingredients (a) to (f), those explained above can be used.

It is possible to further mix therein any of the optional ingredients.

The above process can be performed without heating. For example, the above process can be performed at ambient temperature (25° C.).

Since the (a) biodegradable liquid fatty material used for the composition according to the present invention is already in the form of a liquid at ambient temperature, it is not necessary to heat the biodegradable fatty material to melt when performing the above process to prepare the composition according to the present invention. Therefore, the process for preparing the composition according to the present invention does not require much energy, and therefore, it is friendly to environments.

### [Use]

The composition according to the present invention is preferably used for cosmetic purposes of keratin fibers. Thus, the composition according to the present invention is preferably a cosmetic composition for keratin fibers, in particular for bleaching or coloring keratin fibers.

As the keratin fibers, mention may be made of hair, eyebrows and eyelashes.

If the composition according to the present invention explained above is used for bleaching keratin fibers such as hair, for example, the composition according to the present invention which includes no dye can be used as a mixture with another composition comprising (g) at least one oxidizing agent explained below.

Alternatively, if the composition according to the present invention is used for dyeing keratin fibers such as hair, the composition according to the present invention which further includes (f) at least one dye can be used as a mixture with another composition comprising (g) at least one oxidizing agent.

The above composition including the (g) at least one oxidizing agent can function as a developer.

The above mixture may be regarded as a ready-to-use composition. For the purposes of the present invention, the expression “ready-to-use composition” is defined herein as a composition to be applied immediately to keratin fibers such as hair. The ready-to-use composition can also be a cosmetic composition for keratin fibers, in particular for bleaching or coloring keratin fibers, such as hair.

The mixing ratio of the composition according to the present invention and another composition is not limited. The mixing ratio may be 1:3 to 3:1, preferably 1:2 to 2:1, and more preferably 1:1, as the weight ratio thereof.

### (Oxidizing Agent)

The developer which can be combined with the composition according to the present invention includes at least one (g) oxidizing agent. If two or more (g) oxidizing agents are used, they may be the same or different.

The (g) oxidizing agent may be chosen from hydrogen peroxide, peroxygenated salts, and compounds capable of producing hydrogen peroxide by hydrolysis. For example, the (g) oxidizing agent can be chosen from hydrogen peroxide, urea peroxide, alkali metal bromates and ferricyanides and persalts such as perborates and persulphates. At least one oxidase enzyme chosen, for example, from laccases, peroxidases and 2-electron oxidoreductases such as uricase may also be used as the (g) oxidizing agent, where appropriate in the presence of the respective donor or co-factor thereof.

In one embodiment, the (g) oxidizing agent is hydrogen peroxide, and the developer is an aqueous hydrogen peroxide solution.

In one embodiment, when the developer is an aqueous hydrogen peroxide solution, the developer may comprise at least one hydrogen peroxide stabilizer, which may be chosen, for example, from alkali metal and alkaline-earth metal pyrophosphates, alkali metal and alkaline-earth metal stannates, phenacetin and salts of acids and of oxyquinoline, for example, oxyquinoline sulphate. In another embodiment, at least one stannate optionally in combination with at least one pyrophosphate is used.

It is also possible to use salicylic acid and its salts, pyridinedicarboxylic acid and its salts, and paracetamol.

In the developer in the form of an aqueous hydrogen peroxide solution, the concentration of the hydrogen peroxide stabilizer may range from 0.0001% to 5% by weight such as from 0.01% to 2% by weight, relative to the total weight of the developer.

In the developer in the form of an aqueous hydrogen peroxide solution, the concentration ratio of the hydrogen peroxide to the possible at least one stabilizer may range from 0.05:1 to 1,000:1, such as from 0.1:1 to 500:1 and further such as from 1:1 to 200:1.

The amount of the (g) oxidizing agent(s) in the developer may be from 0.1% by weight or more, preferably 0.5% by weight or more, and more preferably from 1% by weight or more, relative to the total weight of the composition.

The amount of the (g) oxidizing agent(s) in the developer may be from 20% by weight or less, preferably from 15% by weight or less, and more preferably from 10% by weight or less, relative to the total weight of the composition.

The amount of the (g) oxidizing agent(s) in the developer may be from 0.1% to 20% by weight, preferably from 0.5% to 15% by weight, and more preferably from 1% to 10% by weight, relative to the total weight of the composition.

### [Kit]

The present invention also relates to a kit for keratin fibers, preferably a cosmetic kit, and more preferably a cosmetic kit for bleaching or dyeing keratin fibers, in particular hair, comprising:
a first compartment comprising a first composition comprising
(a) at least one biodegradable liquid fatty material;
(b) at least one alkaline agent;
(c) at least one thickener; and
(d) water, and optionally (e) at least one non-ionic surfactant and/or (f) at least one dye,
wherein
the amount of the (a) biodegradable liquid fatty material(s) is 10% by weight or more, preferably 30% by weight or more, and more preferably 50% by weight or more, relative to the total weight of the first composition,
and
a second compartment comprising a second composition comprising
(g) at least one oxidizing agent.

For the above ingredients (a) to (g), those explained above can be used.

It is possible to use the kit by, for example, dispensing or discharging the first composition from the first compartment, while dispensing or discharging the second composition from the second compartment, followed by treating keratin fibers such as hair with the mixture of the first and second compositions.

The mixture of the first and second compositions may be regarded as the ready-to-use composition as explained above.

The mixing ratio of the first and second compositions is not limited. The mixing ratio may be 1:3 to 3:1, preferably 1:2 to 2:1, and more preferably 1:1, as the weight ratio thereof.

### [Process]

The present invention also relates to a process, preferably a cosmetic process, and more preferably a cosmetic process for bleaching or dyeing keratin fibers, in particular hair, comprising the steps of:
(1) mixing a first composition and a second composition to prepare a mixture,
   wherein
   the first composition comprises
   (a) at least one biodegradable liquid fatty material,
   (b) at least one alkaline agent,
   (c) at least one thickener, and
   (d) water, and optionally (e) at least one non-ionic surfactant and/or (f) at least one dye,
   wherein
   the amount of the (a) biodegradable liquid fatty material(s) is 10% by weight or more, preferably 30% by weight or more, and more preferably 50% by weight or more, relative to the total weight of the first composition,
   and
   the second composition comprises
   (g) at least one oxidizing agent;
and
(2) applying the mixture to the keratin fibers.

For the above ingredients (a) to (g), those explained above can be used.

The mixture of the first and second compositions may be regarded as the ready-to-use composition as explained above.

The mixing ratio of the first and second compositions is not limited. The mixing ratio may be 1:3 to 3:1, preferably 1:2 to 2:1, and more preferably 1:1, as the weight ratio thereof.

It is preferable that the process according to the present invention further comprise a step of washing, with or without drying, keratin fibers before and/or after the step of applying the mixture of the first and second compositions, as a ready-to-use composition, onto the keratin fibers.

The step of applying the ready-to-use composition onto the keratin fibers can be performed by a conventional applicator such as a brush, or even by the hands.

The keratin fibers to which the ready-to-use composition has been applied can be left for an appropriate time which is required to treat the keratin fibers. The time length for the treatment is not limited, but it may be from 1 minute to 1 hour, preferably 1 minute to 30 minutes, and more preferably 1 minute to 15 minutes. For example, the time for dyeing the keratin fibers may be from 1 to 20 minutes, preferably 5 to 15 minutes.

The keratin fibers may be treated at room temperature. Alternatively, the keratin fibers can be heated at 25° C. to 65° C., preferably 30° C. to 60° C., more preferably 35° C. to 55° C., and even more preferably 40° C. to 50° C., before and/or during and/or after the step of applying the ready-to-use composition onto the keratin fibers.

### EXAMPLES

The present invention will be described in a more detailed manner by way of examples. However, these examples should not be construed as limiting the scope of the present invention. The examples below are presented as non-limiting illustrations in the field of the present invention.

### Examples 1-3 and Comparative Example 1

The following compositions according to Examples 1-3 and Comparative Example 1, shown in Table 1, were prepared by mixing the ingredients shown in Table 1. The numerical values for the amounts of the ingredients shown in Table 1 are all based on “% by weight” as raw materials.

TABLE 1

| | | | | Comp. |
|---|---|---|---|---|
| | Ex. 1 | Ex. 2 | Ex. 3 | Ex. 1 |
| Coco-Glucoside | 1.30 | 1.30 | 1.30 | 1.30 |
| Water | qsp 100 | qsp 100 | qsp 100 | qsp 100 |
| Hydroxyethylcellulose | 0.5 | 0.5 | 0.5 | 0.5 |
| Hydroxypropyl Guar | 0.3 | 0.3 | 0.3 | 0.3 |
| Sunflower Seed Oil | 60.0 | — | — | — |
| Ethylhexyl Palmitate | — | 60.0 | — | — |
| C15-C19 alkane | — | — | 60.0 | — |
| Mineral Oil | — | — | — | 60.0 |
| Ethanolamine | 4.26 | 4.26 | 4.26 | 4.26 |
| Bleach Test | A | A | A | A |

### [Evaluations]

### (Bleach Test)

Each of the compositions according to Examples 1-3 and Comparative Example 1 was mixed with the developer of the formulation shown in Table 2 below. The mixing weight ratio of the composition and the developer was 1 to 1.

TABLE 2

| | wt % |
|---|---|
| Cetearyl Alcohol | 6 |
| Steareth-20 | 5 |
| Mineral Oil | 25 |
| PEG-4 Rapeseedamide | 1.3 |
| Tocopherol | 0.1 |
| Glycerin | 0.5 |
| Hexadimethrine Chloride (ca. 60%) | 0.25 |
| Tetrasodium Pyrophosphate | 0.04 |
| Hydrogen Peroxide (ca. 50%) | 18 |
| Tetrasodium Etidronate (ca. 30%) | 0.2 |
| Sodium Salicylate | 0.035 |
| Polyquaternium-6 (ca. 40%) | 0.5 |
| Phosphoric Acid | qs. pH 2.2 |
| Water | qsp. 100 |

3g of the mixture thus obtained was applied on 1 g of a tress of natural Chinese natural black hair for 50 minutes at 27° C., followed by washing with water, shampooing, rinsing once and drying the tress.

The difference in color of the tress before and after the above bleaching process was evaluated by using Minolta CM-3600A. ΔE* was calculated by the following formula (1), based on CIE1976.

$$Δ {E}_{i}^{*} = {\{ {( {L}_{i} - {L}_{0} )}^{2} + {( {a}_{i} - {a}_{0} )}^{2} + {( {b}_{i} - {b}_{0} )}^{2} \}}^{1 / 2} \qquad ( 1 )$$

In the formula (1), (Lᵢ, aᵢ, bᵢ) refer to the (L*, a*, b*) values of the hair tress after bleaching, and (L₀, a₀, b₀) refer to the (L*, a*, b*) values of the hair tress before bleaching.

ΔE* values were scored in accordance with the following criteria. The larger ΔE* is, the more bleaching there is.
A: >12
B: <8-12
C: 4-8
D: <4

The results are shown in Table 1.

It was found that the compositions according to Examples 1-3 including biodegradable liquid fatty material provided sufficient bleaching effects which are comparable with that by the composition according to Comparative Example 1 including mineral oil which has poor biodegradability.

1. A composition for keratin fibers, comprising:
(a) at least one biodegradable liquid fatty material;
(b) at least one alkaline agent;
(c) at least one thickener; and
(d) water,
wherein
the amount of the (a) biodegradable liquid fatty material(s) is 10% by weight or more, preferably 30% by weight or more, and more preferably 50% by weight or more, relative to the total weight of the composition.

2. The composition according to claim 1, wherein the (a) biodegradable liquid fatty material is selected from oils derived from plants, ester oils, and mixtures thereof.

3. The composition according to claim 2, wherein the oils derived from plants comprises hydrocarbon oils, preferably alkanes, and more preferably C15-19 alkane.

4. The composition according to any one of claims 1 to 3, wherein the amount of the (a) biodegradable liquid fatty material(s) in the composition is from 10% to 80% by weight, preferably from 30% to 75% by weight, and more preferably from 50% to 70% by weight, relative to the total weight of the composition.

5. The composition according to any one of claims 1 to 4, wherein the (b) alkaline agent is selected from ammonia, alkanolamines, derivatives thereof, and salts thereof.

6. The composition according to claim 5, wherein the alkanolamine is selected from the group consisting of monoethanolamine, diethanolamine, triethanolamine, monoisopropanolamine, diisopropanolamine, N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethylamino)methane, and a mixture thereof.

7. The composition according to any one of claims 1 to 6, wherein the amount of the (b) alkaline agent(s) in the composition is from 0.1% to 20% by weight, preferably from 0.5% to 15% by weight, and more preferably from 1% to 10% by weight, relative to the total weight of the composition.

8. The composition according to any one of claims 1 to 7, wherein the (c) thickener is selected from polysaccharides.

9. The composition according to any one of claims 1 to 8, wherein the (c) thickener is selected from cellulose derivatives, modified guar gums, and mixtures thereof, more preferably hydroxyalkyl celluloses, guar gums modified with C₁-C₆ hydroxyalkyl groups, and mixtures thereof, and even more preferably hydroxyethylcellulose, hydroxypropyl guar, and mixtures thereof.

10. The composition according to any one of claims 1 to 9, wherein the amount of the (c) thickener(s) in the composition is from 0.01% to 10% by weight, preferably from 0.05% to 5% by weight, and more preferably from 0.1% to 1% by weight, relative to the total weight of the composition.

11. The composition according to any one of claims 1 to 10, wherein the amount of the (d) water in the composition is from 10% to 50% by weight, preferably from 15% to 45% by weight, and more preferably from 20% to 40% by weight, relative to the total weight of the composition.

12. The composition according to any one of claims 1 to 9, wherein the composition further comprises (e) at least one non-ionic surfactant, preferably at least one liquid non-ionic surfactant, and more preferably selected from liquid alkyl polyglucosides.

13. The composition according to claim 11 or 12, wherein the amount of the (e) non-ionic surfactant(s) in the composition is from 0.1% to 15% by weight, preferably from 0.5% to 10% by weight, and more preferably from 1% to 5% by weight or more, relative to the total weight of the composition.

14. The composition according to any one of claims 1 to 13, further comprising (f) at least one dye.

15. A process for keratin fibers comprising the steps of:
(1) mixing a first composition and a second composition to prepare a mixture,
wherein
the first composition comprises
(a) at least one biodegradable liquid fatty material,
(b) at least one alkaline agent,
(c) at least one thickener, and
(d) water, and optionally (e) at least one non-ionic surfactant and/or (f) at least one dye,
wherein
the amount of the (a) biodegradable liquid fatty material(s) is 10% by weight or more, preferably 30% by weight or more, and more preferably 50% by weight or more, relative to the total weight of the first composition, and
the second composition comprises
(g) at least one oxidizing agent;
and
(2) applying the mixture to the keratin fibers.
